(19) United States
(12) Patent Application Publication
(10) Pub. No.: US 2024/0024221 A1
(43) Pub. Date: Jan. 25, 2024

(54) COMPOSITION FOR BRIGHTENING AND/OR WHITENING KERATIN MATERIALS

(71) Applicant: L'OREAL, Paris (FR)

(72) Inventors: Yue WANG, Shanghai (CN); Xiaomin WENG, Shanghai (CN); Ji SONG, Shanghai (CN)

(73) Assignee: L'OREAL, Paris (FR)

(21) Appl. No.: 18/258,480

(22) PCT Filed: Dec. 22, 2020
(86) PCT No.: PCT/CN2020/138268

§ 371 Date: Jun. 20, 2023

(51) Int. Cl. A61K 8/67; A61K 8/34; A61Q 19/02

(52) U.S. Cl. CPC A61K 8/675; A61K 8/345; A61Q 19/02; A61K 2800/30

(57) ABSTRACT

Provided herein is a composition for brightening and/or whitening keratin materials, comprising in a hydrophilic phase: (i) at least one glycol with a log P being from −0.5 to 4; (ii) at least one surfactant with a HLB value of 8-20 at the temperature of 25° C.; and (iii) at least one brightening and/or whitening ingredient selected from vitamin B3 and derivatives thereof, wherein the composition is oil-free. Also provided herein is to a non-therapeutic method for brightening and/or whitening keratin materials, comprising applying said composition to the keratin materials, and use of a combination of at least one glycol with a log P being from −0.5 to 4 and at least one surfactant with HLB of 10-20 for improving penetration of a brightening and/or whitening ingredient selected from vitamin B3 and derivatives thereof into keratin materials.

### TECHNICAL FIELD

The present invention relates to a composition and use thereof. In particular, the present invention relates to a composition for brightening and/or whitening keratin materials, in particular the skin. The present invention also relates to a non-therapeutic method for brightening and/or whitening keratin materials, and use of a combination of at least one glycol with a log P being from −0.5 to 4 and at least one surfactant with a HLB value of 8-20 at the temperature of 25° C. for improving penetration of a brightening and/or whitening ingredient selected from vitamin B3 and derivatives thereof into keratin materials.

### BACKGROUND ART

Human skin colour depends on various factors, and in particular on the seasons of the year, on race and on gender; it is mainly determined by the nature and the concentration of melanin produced by melanocytes. Melanocytes are specialized cells which synthesize melanin by means of specific organelles, melanosomes. In addition, at various times in their life, certain individuals experience the appearance of dark and/or coloured spots on the skin and more especially on the hands, which give the skin heterogeneity.

For various reasons associated in particular with greater comfort of use (softness, emollience and the like), current compositions for caring for and/or making up keratin materials, in particular the skin, are usually in the form of an emulsion of the oil-in-water (O/W) type consisting of an aqueous dispersing continuous phase and an oily dispersed discontinuous phase, or of an emulsion of the water-in-oil (W/O) type consisting of an oily dispersing continuous phase and an aqueous dispersed discontinuous phase.

O/W emulsions are the ones most sought in the cosmetics field, since they comprise an aqueous phase as the external phase, which gives them, when applied to the skin, a fresher, less greasy and lighter feel than W/O emulsions.

However, due to their natural feature, the conventional oil-in-water emulsions are not totally satisfying, in particular in terms of brightening and/or whitening of the skin.

Efforts have been made to introduce brightening and/or whitening ingredients such as niacinamide or its derivative into cosmetic products. However, for many products for brightening and/or whitening the skin, it is difficult for the brightening and/or whitening ingredients contained to penetrate into the skin.

For compositions containing brightening and/or whitening ingredients, penetration of the active ingredients to the stratum corneum is one of the most important properties.

There is thus still a need to formulate a composition for brightening and/or whitening the skin, which has an improved effect in terms of penetration of the brightening and/or whitening ingredient such as niacinamide or its derivative contained into the stratum corneum.

### SUMMARY OF THE INVENTION

The inventors have now discovered that it is possible to formulate such compositions having an improved effect in terms of penetration of the cosmetic active ingredient such as niacinamide or its derivative contained to the stratum corneum.

Accordingly, in a first aspect, the present invention provides a composition for brightening and/or whitening keratin materials, comprising in a hydrophilic phase:
(i) at least one glycol with a log P being from −0.5 to 4;
(ii) at least one surfactant with a HLB value of 8-20 at the temperature of 25° C.; and
(iii) at least one brightening and/or whitening ingredient selected from vitamin B3 and derivatives thereof,
wherein the composition is oil-free.

The composition of the present invention can be in the form of a liquid, for example, a toner or a serum.

In a second aspect, the present invention provides a non-therapeutic method for brightening and/or whitening keratin materials, comprising applying the composition according to the first aspect of the present invention to the keratin materials.

It was found that vitamin B3 and/or its derivative contained in the composition according to the present invention can easily penetrate into the stratum corneum.

In a third aspect, the present invention provides use of a combination of at least one glycol with a log P being from −0.5 to 4 and at least one surfactant with a HLB value of 8-20 at the temperature of 25° C. for improving penetration of a brightening and/or whitening ingredient selected from vitamin B3 and derivatives thereof into keratin materials.

Other subjects and characteristics, aspects and advantages of the present invention will emerge even more clearly from the detailed description and the examples that follow.

### BRIEF DESCRIPTION OF THE DRAWINGS

Implementations of the present invention will now be described, by way of example only, with reference to the attached figures, wherein:

FIG. 1 shows the penetration profile of niacinamide for the compositions of invention formulas 1-2 and comparative formula 1.

FIG. 2 shows the penetration profile of niacinamide for the compositions of invention formulas 3-4 and comparative formula 1.

FIG. 3 shows the penetration profile of niacinamide for the compositions of invention formulas 5-6 and comparative formula 1.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

All percentages in the present invention refer to weight percentage, unless otherwise specified.

For the purposes of the present invention, the term “keratin materials” is intended to cover human skin, mucous membranes such as the lips. Facial skin is most particularly considered according to the present invention.

By “oil-free”, it means that no oil is added on purpose and the oil content of the composition is less than 0.5% by weight, relative to the total weight of the composition. In particular, there is no oil in the composition.

According to the first aspect, the present invention provides a composition for brightening and/or whitening keratin materials, comprising in a hydrophilic phase:
(i) at least one glycol with a log P being from −0.5 to 4;
(ii) at least one surfactant with a HLB value of 8-20 at the temperature of 25° C.; and
(iii) at least one brightening and/or whitening ingredient selected from vitamin B3 and derivatives thereof;
wherein the composition is oil-free.

Hydrophilic Phase

According to the first aspect, the composition of the present invention comprises a hydrophilic phase.

The hydrophilic phase comprises water.

Preferably, the hydrophilic phase comprises at least one organic solvent miscible with water (at room temperature 25° C.) such as for example monoalcohols having from 2 to 6 carbon atoms such as ethanol, isopropanol, triols such as glycerin.

Advantageously, water is present in the composition of the present invention in an amount ranging from 60% to 90% by weight, relative to the total weight of the composition.

Glycol

According to the first aspect, the composition of the present invention comprises at least one glycol with a log P being from −0.5 to 4.

A log P value is a value for the base-ten logarithm of the apparent octan-1-ol/water partition coefficient. The log p values are known and are determined by a standard test which determines the concentration of the glycol in octan-1-ol and water. The log P may be calculated according to the method described in the article by Meylan and Howard: Atom/Fragment contribution method for estimating octanol-water partition coefficients, J. Pharm. Sci., 84: 83-92, 1995. This value may also be calculated using numerous commercially available software packages, which determine the log P as a function of the structure of a molecule. By way of example, mention may be made of the Epiwin software from the United States Environmental Agency.

The values may especially be calculated using the ACD (Advanced Chemistry Development) Solaris software V4.67; they may also be obtained from Exploring QSAR: hydrophobic, electronic and steric constants (ACS professional reference book, 1995). There is also an Internet site which provides estimated values (address: http://esc.syrres.com/interkow/kowdemo.htm).

Preferably, the glycol is selected from C4-C10 glycol.

More preferably, the glycol is selected from C4-C8 glycol.

Thus, in some preferred embodiments, the composition of the present invention comprises at least one C4-C10 glycol, preferably C4-C8 glycol.

As examples of C4-C10 glycols that can be used in the composition of the present invention, mention can be made to butylene glycol, pentylene glycol, hexylene glycol, dipropylene glycol, heptanediol, octanediol, nonanediol, and decylene glycol.

In the present invention, the definition of glycols includes all possible isomers. For example, pentylene glycol comprises 1,5-pentylene glycol, 2,4-pentylene glycol, etc.

In a more preferred embodiment, the composition comprises a combination of butylene glycol, pentylene glycol and dipropylene glycol.

Advantageously, the glycol is present in an amount ranging from 2% to 40% by weight, preferably from 5% to 30% by weight, more preferably from 8% to 20% by weight, relative to the total weight of the composition.

Surfactants

According to the first aspect, the composition of the present invention comprises at least one surfactant with a HLB value of 8-20 at the temperature of 25° C.

The HLB (hydrophile-lipophile balance) value is defined according to Griffin's method in J. Ploughshare. Cosm. Chem. 1954 (volume 5), pages 249-256.

Advantageously, the surfactant has a HLB value of 10-15, preferably 12-15, at the temperature of 25° C.

Preferably, the surfactant is selected from non-ionic surfactants, anionic surfactants, amphoteric or zwitterionic surfactant, or a mixture thereof.

Non-ionic surfactants that are suitable to the present composition may be selected from:
polyoxyethylenated sorbitol fatty esters such as the product sold under the name TWEEN 20 by ICI;
polyoxyethylenated fatty alcohols such as the product sold under the name REMCOPAL 21912 AL by Gerland;
polyoxyethylenated alkylphenols such as the product sold under the name TRITON X 100 by Rohm-Haas; and
condensates of ethylene oxide and of propylene oxide such as those sold under the names SYNPERONIC PE by ICI and in particular those referenced L 31, L 64, F 38, F 88, L 92, P 103, F 108 and F 127;
esters of fatty acids and glycerol or polyglycerol, preferably esters of C₆-C₃₀, more preferably C₈-C₁₆, fatty acids, saturated or unsaturated, and glycerol or polyglycerol. Mentions may be made of for example glyceryl stearate, glyceryl isostearate, polyglyceryl-3 diisostearate, glyceryl caprylate, polyglyceryl-4 caprate, polyglyceryl-10 laurate, or a mixture thereof. Such products are available on the market, for example, a mixture of glyceryl stearate and PEG-100 stearate sold under the tradename Simulsoi 165 sold by the company Seppic, or the product polyglyceryl-4 caprate sold under the tradename Tegosoft® PC 41 by the company Evonik Goldshmidt.
ethers of polyethylene glycol and/or of polypropylene glycol, and of glycerol such as glycereth-26 and PPG-24 glycereth-24;
esters derived from the reaction a) of fatty acids and b) polyethylene glycol and/or polypropylene glycol glycerol ethers such as, for example, glycereth-7, or glycereth-25 PCA isostearate;
esters of sucrose and of fatty acids comprising from 12 to 30 carbon atoms, in particular 14 to 20 carbon atoms, said esters possibly comprising from 2 to 5 fatty chains, such as for example sucrose distearate, sucrose tristearate and sucrose palmitate;
alkylpolyglucosides, preferably those that contain an alkyl group comprising from 6 to 30 carbon atoms and preferably from 8 to 16 carbon atoms, and that contain a hydrophilic group (glucoside) preferably comprising 1.2 to 3 sugar units. Mention may be made, for example, of decyl glucoside (Alkyl-C₉/C₁₁-polyglucoside (1.4)) such as the product sold under the name Plantacare® 2000 UP by Cogins (BASF), products sold under the name MYDOL 10 ® by Kao Chemicals, the product sold under the name PLANTAREN® 2000 UP by Cognis, and the product sold under the name ORAMIX® NS 10 by Seppic; caprylyl/capryl glucoside such as the product sold under the name ORAMIX® CG 110 by Seppic; laurylglucoside such as the products sold under the names PLANTAREN® 1200 N and PLANTACARE® 1200 by Cognis; and cocoglucoside such as the product sold under the name PLANTACARE® 818/UP by Cognis, cetostearylglucoside optionally as a mixture with cetostearyl alcohol sold, for example, under the name MONTANOV 68 by Seppic under the name TEGO® CARE CG90 by Goldschmidt and under the name EMULGADE KE3302 by Henkel; arachidylglucoside, for example in the form of the mixture of arachidyl and behenyl alcohols and of arachidylglucoside sold under the name MONTANOV 202 by Seppic; cocoylethylglucoside, for example in the form of the (35/65) mixture with cetyl and stearyl alcohols, sold under the name MONTANOV 82 by Seppic and mixtures thereof.

Mention may in particular be made, among anionic surfactants, of:
alkyl sulphates, alkyl ether sulphates and their salts, in particular their sodium salts, such as the Sodium Laureth Sulphate/Magnesium Laureth Sulphate/Sodium Laureth-8 Sulphate/Magnesium Laureth-8 Sulphate mixture sold under the name TEXAPON ASV by Henkel; sodium lauryl ether sulphate (C₁₂₋₁₄ 70/30) (2.2 EO), sold under the names SIPON AOS 225 or TEXAPON® N702 PASTE by Henkel; ammonium lauryl ether sulphate (C₁₂₋₁₄ 70/30) (3 EO) sold under the name SIPON LEA 370 by Henkel; ammonium alkyl (C_{12-C14}) ether (9 EO) sulphate, sold under the name RHODAPEX® AB/20 by Rhodia Chimie;
alkyl sulphoacetates, such as that sold under the name LATHANOL® LAL by Stepan;
alkyl sulphosuccinates, for example the oxyethylenated (3 EO) lauryl alcohol (C₁₂/C₁₄ 70/30) monosulphosuccinate sold under the names SETACIN 103 SPECIAL or REWOPOL SB-FA 30 K 4 by Witco, the disodium salt of a hemisulphosuccinate of C₁₂-C₁₄ alcohols sold under the name SETACIN F SPECIAL PASTE by Zschimmer Schwarz, the oxyethylenated (2 EO) disodium oleamidosulphosuccinate sold under the name STANDAPOL SH 135 by Henkel, the oxyethylenated (5 EO) lauramide monosulphosuccinate sold under the name LEBON A-5000 by Sanyo, the disodium salt of oxyethylenated (10 EO) lauryl citrate monosulphosuccinate sold under the name REWOPOL® SB CS 50 by Witco or the disodium salt of ricinoleic acid monoethanolamide monosulphosuccinate sold under the name REWODERM® S 1333 by Witco;
polypeptides which are obtained, for example, by condensation of a fatty chain with cereal amino acids and in particular wheat and oat amino acids, such as, for example, the potassium salt of lauroyl hydrolysed wheat protein sold under the name AMINOFOAM® W OR by Croda, the triethanolamine salt of cocoyl hydrolysed soya protein sold under the name MAY-TEIN SY by Maybrook, the sodium salt of lauroyl oat amino acids sold under the name PROTEOL™ OAT by Seppic, the hydrolysate of collagen grafted to coconut fatty acid sold under the name GELIDERM 3000 by Deutsche Gelatine or the soya proteins acylated with hydrogenated coconut acids sold under the name PROTEOL VS 22 by Seppic;
amino acid derivatives, for example salts of amino acid, for example among sarcosinates and in particular acylsarcosinates, such as sodium lauroyl sarcosinate, sold under the name SARKOSYL NL 97 by Ciba or sold under the name ORAMIX™ L 30 by Seppic, sodium myristoyl sarcosinate, sold under the name NIKKOL SARCOSINATE MN by Nikkol, or sodium palmitoyl sarcosinate, sold under the name NIKKOL SARCOSINATE PN by Nikkol; alaninates, such as sodium N-lauroyl-N-methylamidopropionate, sold under the name SODIUM NIKKOL ALANINATE LN 30 by Nikkol or sold under the name ALANONE ALE by Kawaken, and triethanolamine N-lauroyl-N-methylalanine, sold under the name ALANONE ALTA by Kawaken; N-acylglutamates, such as triethanolamine monococoyl glutamate, sold under the name ACYLGLUTAMATE CT-12 by Ajinomoto, and triethanolamine lauroyl glutamate, sold under the name ACYLGLUTAMATE LT-12 by Ajinomoto; aspartates, such as the mixture of triethanolamine N-lauroyl aspartate and of triethanolamine N-myristoyl aspartate sold under the name ASPARACK LM-T52 by Mitsubishi; or glycine derivatives, such as sodium N-cocoyl glycinate and potassium N-cocoyl glycinate, for example the products sold under the names AMILITE GCS-12 and AMILITE GCK-12 by Ajinomoto; lysinates, such as sodium dilauramidoglutamide lysine, for example the product sold under the name PELLICER L-30 by ASAHI KASEI;
sulphonates, for example α-olefin sulphonates, such as the sodium α-olefin (C14-16) sulphonate sold under the name BIO-TERGE® AS-40 by Stepan, sold under the names WITCONATE AOS PROTÉGÉ and SULFRAMINE AOS PH 12 by Witco or sold under the name BIO-TERGE® AS-40 CG by Stepan or the sodium secondary olefin sulphonate sold under the name HOSTAPUR® SAS 30 by Clariant; or linear alkylarylsulphonates, such as the sodium xylenesulphonate sold under the names MANROSOL SXS30, MANROSOL SXS40 or MANROSOL SXS93 by Manro;
isethionates, in particular acyl isethionates, such as sodium cocoyl isethionate, for example the product sold under the name JORDAPON CI P by Jordan.

Mention may in particular be made, among amphoteric or zwitterionic surfactants, of:
alkylamido alkylamine derivatives, such as N-disodium N-cocoyl-N-carboxymethoxyethyl-N-(carboxymethyl)ethylenediamine (CTFA name: Disodium cocoamphodiacetate), sold as a saline aqueous solution under the name MIRANOL C2M CONC NP by Rhodia Chimie; N-sodium N-cocoyl-N-hydroxyethyl-N-(carboxymethyl)ethylene-diamine (CTFA name: sodium cocoamphoacetate) and the mixture of coconut acid ethanolamides (CTFA name: Cocamide DEA);
betaines, such as, for example, coco betaine, such as the product sold under the name DEHYTON® AB-30 by Henkel, lauryl betaine, such as the product sold under the name GENAGEN® KB by Clariant, and the product sold under the name EMPIGEN BB/LS by INNOSPEC ACTIVE CHEMICALS, oxyethylenated (10 EO) lauryl betaine, such as the product sold under the name LAURYL ETHER (10 EO) BETAINE by Shin Nihon Rica, or oxyethylenated (10 EO) stearyl betaine, such as the product sold under the name STEARYL ETHER (10 EO) BETAINE by Shin Nihon Rica;
alkyl amidopropyl betaines and their derivatives, such as, for example, cocamidopropyl betaine, sold under the name LEBON 2000 HG by Sanyo or sold under the name EMPIGEN® BB by Albright & Wilson, lauramidopropyl betaine, sold under the name REWOTERIC AMB12P® by Witco, such as cocamidopropyl betaine, for example the products sold under the names TEGO BETAINE by Goldschmidt;
imidazoline derivatives, such as the product sold under the name CHIMEXANE HD by Chimex; and
their mixtures.

Preferably, the surfactant is selected from esters of fatty acids and glycerol or polyglycerol, salts of amino acid; betaines, and a mixture thereof.

More preferably, the surfactant is selected from esters of C₆-C₃₀, preferably C₈-C₁₆, saturated or unsaturated fatty acids and glycerol or polyglycerol, lysinates, betaines, and a mixture thereof.

According to a preferred embodiment, the surfactant is selected from sodium dilauramidoglutamide lysine, polyglyceryl-4 caprate, polyglyceryl-10 laurate, lauryl betaine, and a mixture thereof.

Advantageously, the surfactant is present in an amount ranging from 0.001% to 3% by weight, preferably from 0.003% to 2% by weight, more preferably from 0.005% to 1% by weight, relative to the total weight of the composition.

Vitamin B3 and Derivatives Thereof

According to the first aspect, the composition of the present invention comprises at least one brightening and/or whitening ingredient selected from vitamin B3 and derivatives thereof.

Niacinamide is also called vitamin B3 and vitamin PP, having the following structure:

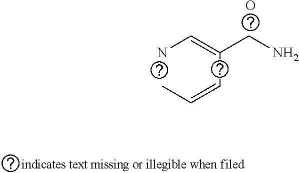

Vitamin B3 derivatives that may be mentioned include, for example, nicotinic acid esters such as tocopherol nicotinate, amides derived from niacinamide by substitution of the hydrogen groups of —CONH₂, products from reaction with carboxylic acids and amino acids, esters of nicotinyl alcohol and of carboxylic acids such as acetic acid, salicyclic acid, glycolid acid or palmitic acid.

Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine, methyl nicotinate and sodium nicotinate.

Other vitamin B3 derivatives that may also be mentioned include its inorganic salts, such as chlorides, bromides, iodides or carbonates, and its organic salts, such as the salts obtained by reaction with carboxylic acids, such as acetate, salicylate, glycolate, lactate, malate, citrate, mandelate, tartrate, etc.

Advantageously, the brightening and/or whitening ingredient selected from vitamin B3 and derivatives thereof is present in an amount ranging from 0.1% to 15% by weight, preferably from 0.5% to 10% by weight, more preferably from 1% to 8% by weight, relative to the total weight of the composition.

Additional Cosmetic Active Ingredients

Depend on the final purpose, the composition according to the present invention can comprises one or more additional cosmetic active ingredients.

As cosmetic active ingredients that may be used in the composition of the present invention, examples that may be mentioned include enzymes; flavonoids; moisturizers anti-inflammatory agents; peeling agents, antiaging agents, antioxidants, depigmenting agents; α-hydroxy acids; tensioning agents; and mixtures thereof.

It is easy for the skilled in the art to adjust the amount of the additional cosmetic active ingredient based on the final use of the composition according to the present invention.

Additional Adjuvants or Additives

The composition of the present invention may comprise conventional cosmetic adjuvants or additives, for instance fragrances, chelating agents (for example, disodium EDTA), preserving agents (for example, hydroxyacetophenone, chlorphenesin and phenoxyethanol) and bactericides, thickeners, fillers, pH regulators (for example citric acid, sodium hydroxide, potassium hydroxide), and mixtures thereof.

The skilled in the art can select the amount of the additional adjuvants or additive so as not to adversely impact the final use of the composition according to the present invention.

According to a particularly preferred embodiment, the present invention provides a composition for brightening and/or whitening keratin materials, comprising in a hydrophilic phase, relative to the total weight of the composition:
(i) from 8% to 20% by weight of a combination of butylene glycol, pentylene glycol and dipropylene glycol;
(ii) from 0.005% to 1% by weight of at least one surfactant selected from sodium dilauramidoglutamide lysine, polyglyceryl-4 caprate, polyglyceryl-10 laurate, lauryl betaine, and a mixture thereof; and
(iii) from 1% to 8% by weight of vitamin B3,
wherein the composition is oil-free.

Galenic Form

The composition of the present invention is preferably in the form of liquid.

Preferably, the composition according to the present invention has a low viscosity.

According to a preferred embodiment, the composition of the present invention has a viscosity below 50 UD (Deviation Units), measured at 25° C. using a Rheomat 180 viscometer equipped with a spindle M1 rotating at 200 rpm.

Method and Use

According to the second aspect, the present invention provides to a non-therapeutic method for brightening and/or whitening keratin materials, comprising applying the composition according to the first aspect of the present invention to the keratin materials.

In particular, the keratin material is intended to mean the skin, especially the face.

According to the third aspect, the present invention provides use of a combination of at least one glycol with a log P being from −0.5 to 4 and at least one surfactant with HLB of 10-20 for improving penetration of a brightening and/or whitening ingredient selected from vitamin B3 and derivatives thereof into keratin materials.

The glycol with a log P being from −0.5 to 4 and the surfactant with a HLB value of 8-20 at the temperature of 25° C. are defined as above.

Preferably, the at least one glycol is selected from C4-C10 glycol, and the at least one surfactant is selected from esters of C6-C30 saturated or unsaturated fatty acids and glycerol or polyglycerol, lysinates, betaines, and a mixture thereof.

More preferably, the at least one glycol is a combination of butylene glycol, pentylene glycol and dipropylene glycol, and the at least one surfactant is selected from sodium dilauramidoglutamide lysine, polyglyceryl-4 caprate, polyglyceryl-10 laurate, lauryl betaine, and a mixture thereof.

The following examples serve to illustrate the present invention without being limiting in nature.

### EXAMPLES

Main raw materials used, trade names and supplier thereof are listed in Table 1.

TABLE 1

| INCI Name | Trade Name | Supplier | HLB |
|---|---|---|---|
| BUTYLENE GLYCOL | 1,3 BUTYLENE GLYCOL | ALZO | — |
| PENTYLENE GLYCOL | A-LEEN 5 | MINASOLVE | — |
| DIPROPYLENE GLYCOL | DIPROPYLENEGLYCOL CARE | BASF | — |
| NIACINAMIDE | NIACINAMIDE PC | DSM NUTRITIONAL | — |
| | | PRODUCTS | |
| HYDROXYACETOPHENONE | SymSave ® H | SYMRISE | |
| POLYGLYCERYL-10 LAURATE | dermofeel ®G 10 L | DR STRAETMANS | 15 |
| | | (EVONIK) | |
| SODIUM | Pellicer ™ L-30 | ASAHI KASEI | 12.6 |
| DILAURAMIDOGLUTAMIDE LYSINE | | | |
| LAURYL BETAINE | EMPIGEN ® BB/LS | INNOSPEC ACTIVE | 15 |
| | | CHEMICALS | |
| POLYSORBATE 20 | TEGO SML 20 MB | EVONIK | 18.6 |
| | | GOLDSCHMIDT | |
| POLYGLYCERYL-2 OLEATE | SUNSOFT Q-17D(G)-C | TAIYO KAGAKU | 8 |
| OCTYLDODECYL XYLOSIDE | FLUIDANOV 20 X | SEPPIC | 7.3 |

### Example 1: Preparation of Compositions

Compositions according to comparative formulas (Comp.) 1-2 and invention formula (Inv.) 1-6 were prepared according to the contents given in Tables 2-3 (the contents are expressed as weight percentages of active material relative to the total weight of each composition, unless otherwise indicated).

TABLE 2

| | Inv. 1 | Inv. 2 | Inv. 3 | Inv. 4 | Comp. 1 |
|---|---|---|---|---|---|
| Ingredients | % by weight | | | | |
| BUTYLENE GLYCOL | 7 | 7 | 7 | 7 | 4.5 |
| PENTYLENE GLYCOL | 3 | 3 | 3 | 3 | 3.5 |
| DIPROPYLENE GLYCOL | 3 | 3 | 3 | 3 | 0 |
| NIACINAMIDE | 5 | 5 | 5 | 5 | 5 |
| WATER | QS100 | QS100 | QS100 | QS100 | QS100 |
| HYDROXYACETOPHENONE | 0.5 | 0.5 | 0.5 | 0.5 | — |
| POLYGLYCERYL-10 LAURATE | 0.005 | 1 | — | — | — |
| SODIUM | — | — | 0.5 | — | — |
| DILAURAMIDOGLUTAMIDE | | | | | |
| LYSINE | | | | | |
| LAURYL BETAINE | — | — | — | 0.01 | — |

Composition of comparative formula 1 does not comprise a surfactant with a HLB value of 8-20 at the temperature of 25° C.

TABLE 3

| | Inv. 5 | Inv. 6 | Comp. 2 |
|---|---|---|---|
| Ingredients | % by weight | | |
| PENTYLENE GLYCOL | 3 | 3 | 3 |
| NIACINAMIDE | 5 | 5 | 5 |
| WATER | QS100 | QS100 | QS100 |
| HYDROXYACETOPHENONE | 0.5 | 0.5 | 0.5 |
| POLYSORBATE 20 | 0.5 | — | — |
| POLYGLYCERYL-2 OLEATE | — | 0.5 | — |
| OCTYLDODECYL XYLOSIDE | — | — | 0.5 |

Composition of comparative formula 2 comprises a surfactant with HLB of 7.3 rather than a surfactant with a HLB value of 8-20 at the temperature of 25° C. The composition obtained is not stable and separated into two phases, it cannot form a uniform liquid.

Preparation Process:

The compositions listed above were prepared as follows, taking the composition of invention formula 1 as an example:
1). dissolving NIACINAMIDE in water with stirring to obtain a main phase;
2). dissolving hydroxyacetophenone in the glycol(s) with stirring until a clear phase is obtained;
3). adding the clear phase comprising hydroxyacetophenone and the glycol(s) in the main phase with stirring until a clear mixture is obtained;
3). adding the surfactant in the clear mixture carefully with stirring to obtain the composition.

### Example 2: Evaluation of Compositions

The penetration of niacinamide in each composition prepared in Example 1 was characterized through In-vitro Skin Permeation Test (Strat-M Test) using Strat-M, which is a synthetic membrane mimicking lipohilic component of the stratum corneum available from the company Merk.

The procedure was as follows:
subjecting a receptor fluid (RF, a PBS solution of pH7.4) to a ultrasonic bath for 15 minutes to remove gas;
installing 3 Franz cells in parallel for each composition on a shelf;
connecting the cells in parallel with a fluidic thermostat in order to homogenize the temperature for each cell, wherein T_{bath}=34° C. and T_{cells}=32° C.;
introducing a magnetic bar in the cells (RF compartment);
marking a line on the sampling port leaving about 1 cm from the sampling port entrance to the line, which is a reference for filling the receptor fluid;
introducing Molykote 111 (a valve lubricant and sealant from the company Dow corning in a 5 mL plastic syringe, spread Molykote 111 with the syringe on the sanded surface of the receptor part of the cell, put the donor compartment on the Franz cell and carry out a complete rotation of 360°, checking that Molykote 111 is well applied homogeneously on the Franz cell edges, withdrawing the donor compartment of the cell and removing the excess of Molykote inside the cell (donor & receiver compartments) with a cotton bud to avoid any contamination;
putting the Strat-M membranes at the center of the cells with shiny side towards donor compartment without touching the edges of the membrane;
aligning the donor compartment and close the Franz cell with a clip without strength;
introducing the receptor fluid through the sampling port and turning on the stirrer at 600 rpm;
flipping upside down the diffusion cell and using a stirring bar to eliminate the bubbles under the membrane;
taking 45 mg of the composition tested and applying the composition tested uniformly on the membrane by a 360 degrees rotation movement and careful tappings, precisely determining the amount of the composition that was applied on the membrane;
sampling 200 μL of receptor fluid using an Hamilton syringe of 250 μL, minimum 3 samplings per hour from 1 hour to 24 hours,
slowly filling the sampling port until reaching the mark with fresh receptor fluid; and
measuring the amount of the active ingredient in the receptor fluid to determine the relationship of the amount of the active ingredient and penetration time.

FIG. 1 shows the penetration profile of niacinamide for the compositions of invention formulas 1-2 and comparative formula 1.

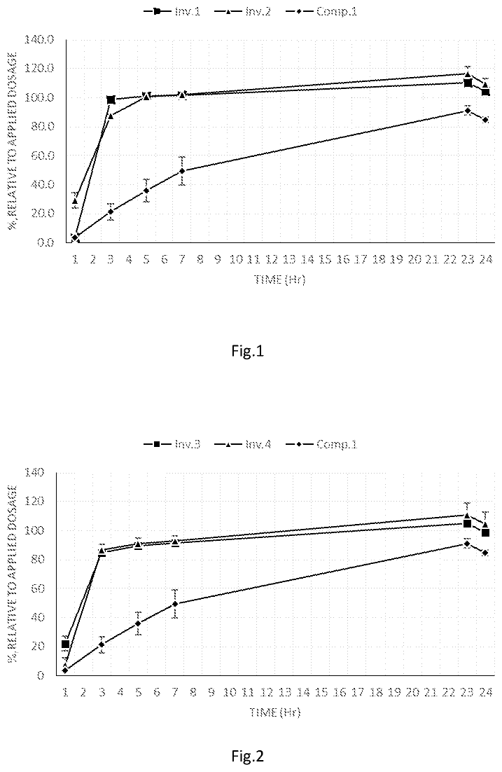

FIG. 2 shows the penetration profile of niacinamide for the compositions of invention formulas 3-4 and comparative formula 1.

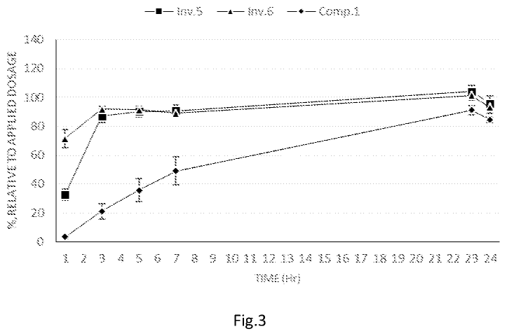

FIG. 3 shows the penetration profile of niacinamide for the compositions of invention formulas 5-6 and comparative formula1.

It can be seen from FIGS. 1-3 that a combination of at least one glycol and at least one surfactant with a HLB value of 8-20 at the temperature of 25° C. can improve the penetration of niacinamide into the skin after topical application.

The viscosity of each composition prepared in Example 1 was measured at 25° C. using a Rheomat 180 viscometer from the company ProRheo equipped with a spindle M1 or M3 rotating at 200 rpm.

The results are as summarized in Table 4.

TABLE 4

| | Inv.1 | Inv.2 | Inv.3 | Inv.4 | Inv.5 | Inv. 6 | Comp.1 | Comp.2 |
|---|---|---|---|---|---|---|---|---|
| Viscosity | 34 | 34 | 34 | 34 | 34 | 34 | 18 | 34 |
| (UD) | (M1) | (M1) | (M1) | (M1) | (M1) | (M1) | (M3) | (M1) |

It can be seen from Table 4 that the compositions according to the present invention have a low viscosity.

1. A composition for brightening and/or whitening keratin materials, comprising in a hydrophilic phase:
(i) at least one glycol with a log P being from −0.5 to 4;
(ii) at least one surfactant with a HLB value of 8-20 at the temperature of 25° C.; and
(iii) at least one brightening and/or whitening ingredient selected from vitamin B3 and derivatives thereof,
wherein the composition is oil-free.

2. The composition according to claim 1, wherein the glycol is selected from the group consisting of C4-C10 glycols.

3. The composition according to claim 1, wherein the glycol is a combination of butylene glycol, pentylene glycol and dipropylene glycol.

4. The composition according to claim 1, wherein the glycol is present in an amount ranging from 2% to 40% by weight relative to the total weight of the composition.

5. The composition according to claim 1, wherein the surfactant is selected from the group consisting of esters of fatty acids and glycerol or polyglycerol, salts of amino acid; betaines, and a mixture thereof.

6. The composition according to claim 1, wherein the surfactant is selected from the group consisting of esters of C6-C30 saturated or unsaturated fatty acids and glycerol or polyglycerol, lysinates, betaines, and a mixture thereof.

7. The composition according to claim 1, wherein the the surfactant is present in an amount ranging from 0.001% to 3% by weight relative to the total weight of the composition.

8. The composition according to claim 1, wherein the brightening and/or whitening ingredient selected from vitamin B3 and derivatives thereof is present in an amount ranging from 0.1% to 15% by weight relative to the total weight of the composition.

9. The composition according to claim 1, comprising in a hydrophilic phase, relative to the total weight of the composition:
(i) from 8% to 20% by weight of a combination of butylene glycol, pentylene glycol and dipropylene glycol;
(ii) from 0.005% to 1% by weight of at least one surfactant selected from the group consisting of sodium dilauramidoglutamide lysine, polyglyceryl-4 caprate, polyglyceryl-10 laurate, lauryl betaine, and a mixture thereof; and
(iii) from 1% to 8% by weight of vitamin B3,
wherein the composition is oil-free.

10. The composition according to claim 1, wherein the hydrophilic phase comprises water.

11. A non-therapeutic method for brightening and/or whitening keratin materials, comprising applying the composition according to claim 1 to the keratin materials.

12. A method to improve penetration of a brightening and/or whitening ingredient into a keratin material, comprising combining at least one glycol with a log P being from −0.5 to 4 and at least one surfactant with a HLB value of 8-20 at the temperature of 25° C. with the brightening and/or whitening ingredient wherein the brightening and/or whitening ingredient is selected from the group consisting of vitamin B3 and derivatives thereof.

13. The method according to claim 12, wherein the at least one glycol is selected from the group consisting of C4-C10 glycols, and the at least one surfactant is selected from the group consisting of esters of C₆-C₃₀ saturated or unsaturated fatty acids and glycerol or polyglycerol, lysinates, betaines, and a mixture thereof.

14. The method according to claim 12, wherein the at least one glycol is a combination of butylene glycol, pentylene glycol and dipropylene glycol, and the at least one surfactant is selected from the group consisting of sodium dilauramidoglutamide lysine, polyglyceryl-4 caprate, polyglyceryl-10 laurate, lauryl betaine, and a mixture thereof.
